(19) United States
(12) Patent Application Publication
(10) Pub. No.: US 2022/0008238 A1
(43) Pub. Date: Jan. 13, 2022

(54) ORTHOSIS DEVICE HAVING METAL WIRE ADJUSTING STRUCTURE

(71) Applicant: YONG TAI GLOBAL CO., LTD., TAINAN CITY (TW)

(72) Inventor: TSENG-CHIU WANG, TAINAN CITY (TW)

(21) Appl. No.: 17/365,082

(22) Filed: Jul. 1, 2021

(30) Foreign Application Priority Data
Jul. 10, 2020 (TW) 109208835

(51) Int. Cl. A61F 5/01

(52) U.S. Cl. CPC A61F 5/0118; A61F 2005/0197

(57) ABSTRACT

An orthosis device having a metal wire adjusting structure includes a palm orthosis body and a forearm orthosis body. The palm orthosis body includes a plurality of fixing portions. A plurality of adjustment seats are connected to the fixing portions, respectively. The adjustment seats each have a first through hole for insertion of a first end of an elastic metal wire. A first adjusting bolt is screwed to the outer side of each of the adjustment seats, so as to tighten and secure the first end of the elastic metal wire. A second end of the elastic metal wire is inserted into a second through hole of a movable seat. A second adjusting bolt is screwed to the outer side of the movable seat, so as to tighten and secure the second end of the elastic metal wire. A finger sleeve is connected to the movable seat.

### FIELD OF THE INVENTION

The present invention relates to an orthosis device, and more particularly to an orthosis device having a metal wire adjusting structure for replacing elastic metal wires of different diameters to generate different elastic restoring forces.

### BACKGROUND OF THE INVENTION

As is known to all, in the case of damages to the joint tissues subsequent to stroke, surgery or traumas, the normal functionality of the hand fingers or the wrist may be altered, with both physical and psychical consequences. In general, it is necessary to use a hand rehabilitation device for rehabilitation.

Taiwan Patent Publication No. 1541011 discloses a hand rehabilitation device, published on Jul. 11, 2016. The hand rehabilitation device includes a palm-shaped plate and a forearm fixing plate. The palm-shaped plate and the forearm fixing plate are connected to each other to form an included angle. The top and one side of the palm-shaped plate are provided with five rods and five finger sleeves for receiving the user's fingers. One end of a pulling rope is connected to each finger sleeve, and the other end of the pulling rope is inserted through a guide groove of each rod and a guide collar and then secured to a collection collar. One end of a connecting rope is fixed to the collection collar. An upper arm fixing plate is provided with a pulling rope adjustment assembly. The other end of the connecting rope is fixed to the pulling rope adjustment assembly. The upper arm fixing plate is attached to the upper arm. The forearm fixing plate is attached to the forearm. All the pulling ropes can be pulled to be tightened or loosened by moving the upper arm or using the other hand to pull the connecting rope, thereby enabling the five fingers to stretch or bend for rehabilitation.

In the above-mentioned patent, the forearm fixing plate and the palm-shaped plate are fixed as a whole, and an included angle between the forearm fixing plate and the palm-shaped plate is less than 180 degrees. However, the included angle is a fixed angle and cannot be adjusted according to the bending angle of the wrist joint of a different user. By pulling a handle fixed to the other end of the connecting rope, all the pulling ropes are pulled to be tightened or loosened, enabling the five fingers to stretch or bend for rehabilitation. Because all the pulling ropes are pulled at a time to adjust the five fingers for rehabilitation, it is unable to adjust different elastic forces for different fingers separately according to the bending degree of different fingers, so it is not ideal for use.

In addition, Taiwan Utility Model Publication No. M516411 discloses a hand dynamic orthosis, published on Feb. 1, 2016. The hand dynamic orthosis includes an orthosis plate. The orthosis plate is provided with a finger traction elastic assembly corresponding to each finger of the human body. Each finger traction elastic assembly includes an elastic line. One end of the elastic line is defined as an elastic positioning end positioned on the surface of the orthosis plate, and the other end of the elastic line is extended with a set length and provided with a strap. After the orthosis plate is worn on the patient's hand, when the finger sleeved with the finger traction elastic assembly is to bend and stretch to pull the elastic positioning end at the other end of the finger traction elastic assembly in an elastic manner, so as to form corresponding elastic traction to bend and stretch the proximal and distal joints of the patient's finger for dynamic rehabilitation of the hand.

In the above-mentioned patent, the orthosis plate includes a palm portion and a wrist portion that are integrally formed. Both the palm portion and the wrist portion are fixed and integrated, and they cannot be adjusted according to the bending angle of the wrist joint of a different user. After the orthosis plate is worn on the patient's hand, when the finger tied to one end of the elastic line of the finger traction elastic assembly is to bend and stretch, the other end of the elastic line of the finger traction elastic assembly will form the elastic traction function for rehabilitation. It can be installed in accordance with the different conditions of the proximal and distal joints of the patient's finger. Besides, in various situations during the training, through an elastic coil (spring), the relative elasticity is adjusted for better rehabilitation. Because the elasticity of the general spring is relatively large, it is not suitable for patients whose fingers cannot be controlled to perform fine adjustment. Moreover, due to the excessive force of the spring, the patient's fingers cannot move at all. It is difficult to achieve the effects of training and rehabilitation. The adjustment of the elasticity of the spring is limited. If it is required for small elasticity, a spring with few coils and a small diameter is replaced. Therefore, it is quite troublesome and time-consuming to disassemble and replace the spring.

Some of the conventional orthosis devices are provided with plastic tubes instead of elastic wires or springs. However, the elastic restoring force of the plastic tube is not good, and its elastic restoring force cannot be adjusted, so it still has some shortcomings in use.

### SUMMARY OF THE INVENTION

In view of the shortcomings of the prior art, the primary object of the present invention is to provide an orthosis device having a metal wire adjusting structure. The orthosis device comprises a palm orthosis body and a forearm orthosis body. The palm orthosis body includes a plurality of fixing portions. Each of the fixing portions is detachably connected with an elastic metal wire. The elastic metal wire is further detachably connected to a movable seat. A finger sleeve is connected to the movable seat. The forearm orthosis body is coupled to the palm orthosis body.

In an embodiment of the present invention, each of the fixing portions is connected with an adjustment seat. The adjustment seat has a first through hole for insertion of a first end of the elastic metal wire. A first adjusting bolt is screwed to an outer side of the adjustment seat for tightening and securing the first end of the elastic metal wire. A second end of the elastic metal wire is inserted into a second through hole of the movable seat. A second adjusting bolt is screwed to an outer side of the movable seat for tightening and securing the second end of the elastic metal wire.

In an embodiment of the present invention, each of the fixing portions is connected with an adjustment seat. The adjustment seat is formed with a first locking groove. A first locking block is detachably engaged in the first locking groove. A first end of the elastic metal wire is inserted and secured to the first locking block. The movable seat is formed with a second locking groove. A second locking block is detachably engaged in the second locking groove. A second end of the elastic metal wire is inserted and secured to the second locking block.

In an embodiment of the present invention, each of the fixing portions is connected with an adjustment seat. The adjustment seat is provided with a first elastic portion that is tapered and has a cut-out area. A first nut is screwed to the first elastic portion. A first end of the elastic metal wire is inserted into the first elastic portion. The first nut is tightened to tightly secure the first end of the elastic metal wire. The movable seat is provided with a second elastic portion. A second nut is screwed to the second elastic portion. A second end of the elastic metal wire is inserted into the second elastic portion. The second nut is tightened to tightly secure the second end of the elastic metal wire.

Preferably, the elastic metal wire is made of iron or steel.

Preferably, the elastic metal wire has a diameter of between 0.8 mm and 2.0 mm.

Preferably, the palm orthosis body is provided with a first coupling portion, the forearm orthosis body is provided with a second coupling portion, and the second coupling portion is coupled to the first coupling portion through a connecting rod.

Preferably, two sides of the forearm orthosis body are provided with fastening straps.

Preferably, the fastening straps are secured with a hook-and-loop tape.

The above technical features have the following advantages:

1. According to different angles of curling the user's fingers, elastic metal wires of different diameters are selected and replaced, so that different elastic restoring forces are generated to cooperate with different fingers for rehabilitation.

2. It can effectively improve some conventional orthosis devices that have poor elastic restoring forces and are unable to adjust the elastic restoring forces.

3. By screwing or unscrewing the first adjusting bolt on the adjustment seat and the second adjusting bolt on the movable seat, the elastic metal wire of a different diameter can be replaced easily and quickly for rehabilitation.

4. The elastic metal wire is made of iron or steel, so it can provide excellent elastic restoring force. Besides, it is not easy to be damaged or deformed to prolong its service life.

### BRIEF DESCRIPTION OF THE DRAWINGS

FIG. 1 is an exploded view according to a first embodiment of the present invention;

FIG. 2 is a cross-sectional view of an adjustment seat according to the first embodiment of the present invention;

FIG. 3 is a cross-sectional view of a movable seat according to the first embodiment of the present invention;

FIG. 4 is a schematic view according to the first embodiment of the present invention when in use;

FIG. 5 is a schematic view of replacing an elastic metal wire with a different diameter according to the first embodiment of the present invention;

FIG. 6 is a partial exploded view according to a second embodiment of the present invention; and

FIG. 7 is a partial exploded view according to a third embodiment of the present invention.

### DETAILED DESCRIPTION OF THE PREFERRED EMBODIMENTS

Embodiments of the present invention will now be described, by way of example only, with reference to the accompanying drawings.

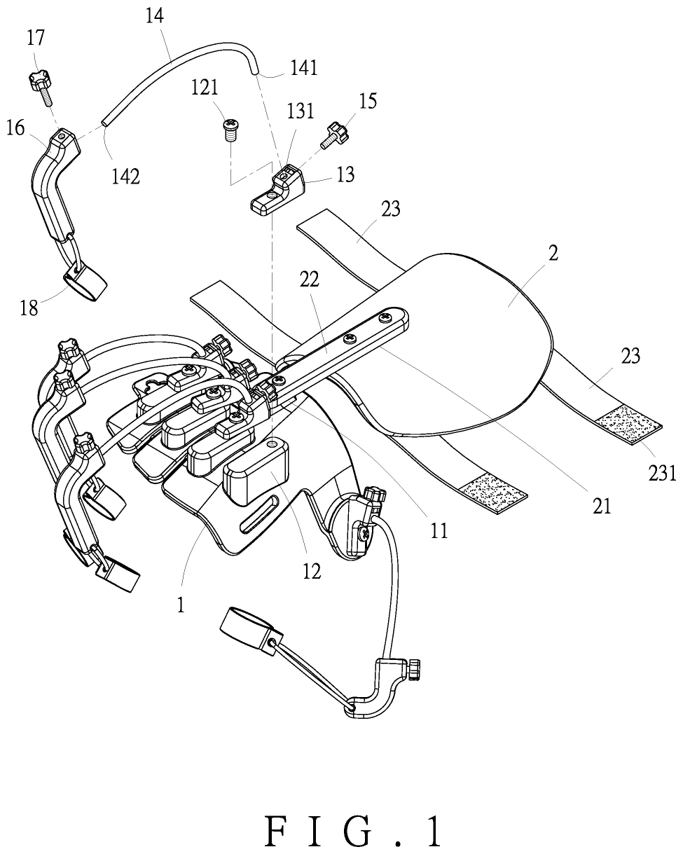

As shown in FIG. 1, an orthosis device having a metal wire adjusting structure according to a first embodiment of the present invention comprises a palm orthosis body 1 and a forearm orthosis body 2.

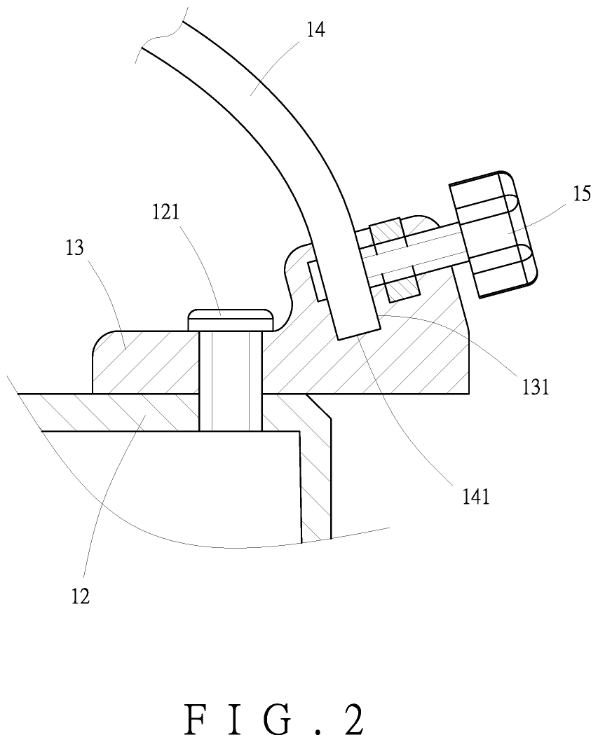

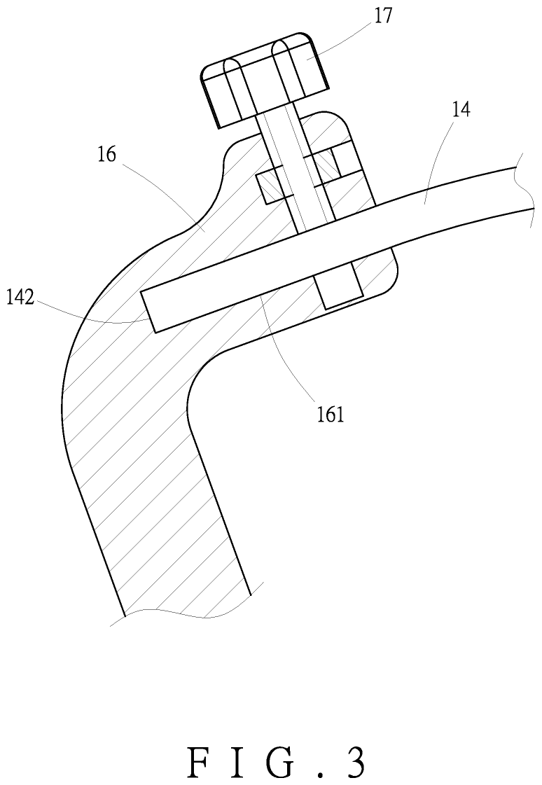

One end of the palm orthosis body 1 is provided with a first coupling portion 11. The other end of the palm orthosis body 1 is provided with a plurality of fixing portions 12. As shown in FIG. 2, a plurality of adjustment seats 13 are connected to the fixing portions 12 by screws 121, respectively. The adjustment seats 13 each have a first through hole 131 for insertion of a first end 141 of an elastic metal wire 14. A first adjusting bolt 15 is vertically screwed to the outer side of each of the adjustment seats 13, so as to tighten and secure the first end 141 of the elastic metal wire 14. The elastic metal wire 14 is made of iron or steel, and its diameter is between 0.8 mm and 2.0 mm. An elastic metal wire 14 with an appropriate diameter is selected and installed according to the requirement of a different elastic restoring force. A second end 142 of the elastic metal wire 14 is inserted into a second through hole 161 of a movable seat 16, as shown in FIG. 3. A second adjusting bolt 17 is vertically screwed to the outer side of the movable seat 16, so as to tighten and secure the second end 142 of the elastic metal wire 14. In addition, a finger sleeve 18 is connected to the movable seat 16 for a corresponding one of a user's fingers to be inserted and positioned in the finger sleeve 18.

The forearm orthosis body 2 is provided with a second coupling portion 21. The second coupling portion 21 is coupled to the first coupling portion 11 through a connecting rod 22, so that the palm orthosis body 1 is coupled to the forearm orthosis body 2. Two sides of the forearm orthosis body 2 are provided with fastening straps 23. The fastening straps 23 are secured with a hook-and-loop tape 231.

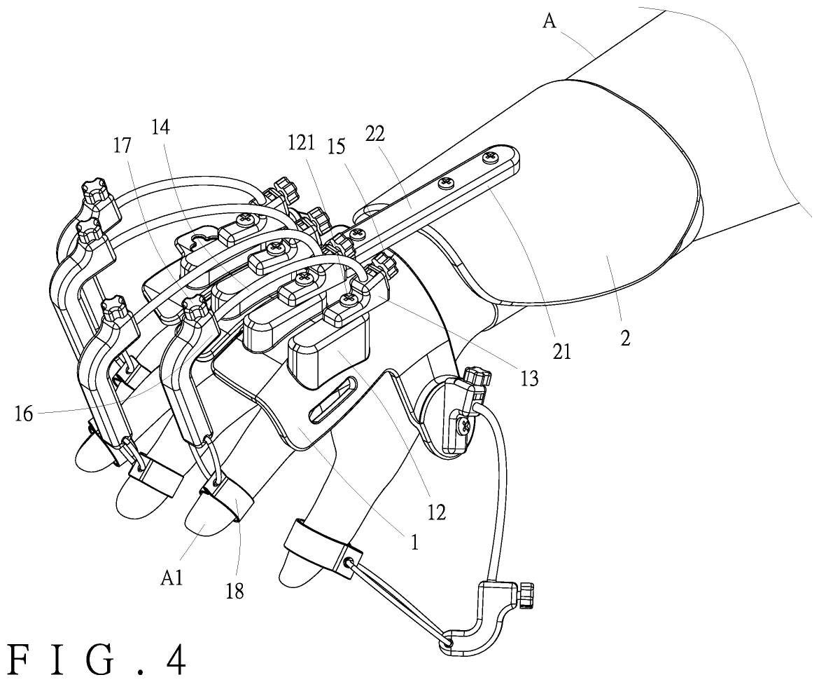

When in use, as shown in FIG. 1 and FIG. 4, the forearm orthosis body 2 is fitted onto a user's forearm A close to the wrist, and the fastening straps 23 on both sides of the forearm orthosis body 2 are wound around the forearm A and secured with the hook-and-loop tape 231, so that the forearm A is positioned in the forearm orthosis body 2. Then, the user's fingers A1 are inserted and positioned in the finger sleeves 18 of the movable seats 16, respectively. The elastic restoring force of the elastic metal wire 14 provides a backward pulling force to the corresponding finger A1 through the second end 142, so as to perform rehabilitation on the finger to prevent the finger A1 from curling and deforming.

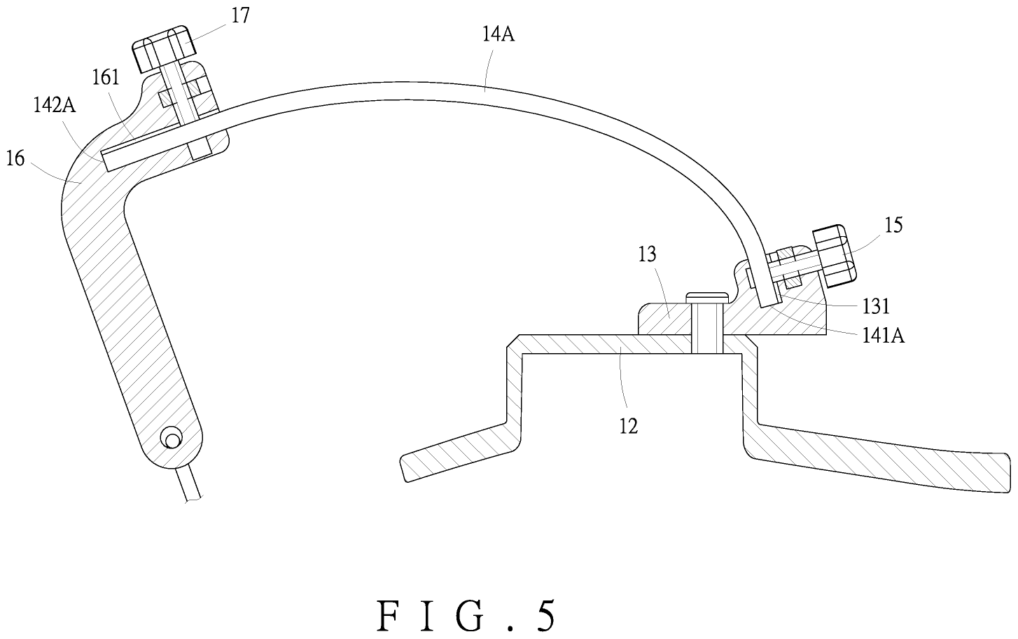

As shown in FIG. 4 and FIG. 5, according to the different degree of rehabilitation of the finger A1, when it is necessary to select a larger or smaller elastic restoring force, the first adjusting bolt 15 on the adjustment seat 13 corresponding to the position of the finger A1 is loosened and the second adjusting bolt 17 on the movable seat 16 corresponding to the position of the finger A1 is loosened, so that the first end 141 and the second end 142 of the elastic metal wire 14 are released, respectively. Then, the elastic metal wire 14 is removed from the adjustment seat 13 and the movable seat 16, and another elastic metal wire 14A with a different diameter is selected according to the degree of rehabilitation of the finger A1. The first end 141A of the elastic metal wire 14A is inserted into the first through hole 131 of the adjustment seat 13, and then the first adjusting bolt 15 is tightened, so as to tightly secure the first end 141A of the elastic metal wire 14A. After that, the second end 142A of the elastic metal wire 14A is inserted into the second through hole 161 of the movable seat 16, and then the second adjusting bolt 17 is tightened, so as to tightly secure the second end 142A of the elastic metal wire 14A. In this way, the elastic metal wire 14A with a different diameter can be easily and quickly replaced for rehabilitation. In the embodiment of the present invention, the elastic metal wire 14A with a smaller diameter is selected for exerting a smaller elastic restoring force to the finger A1, in cooperation with the degree of rehabilitation of the finger A1.

Thus, according to the different angle of curling of the finger A1, an elastic metal wire 14A with a different diameter can be replaced, so that a different elastic restoring force is generated to cooperate with a different finger A1 for rehabilitation. By screwing or unscrewing the first adjusting bolt 15 on the adjustment seat 13 and the second adjusting bolt 17 on the movable seat 16, the elastic metal wire 14 with a different diameter can be replaced easily and quickly for rehabilitation.

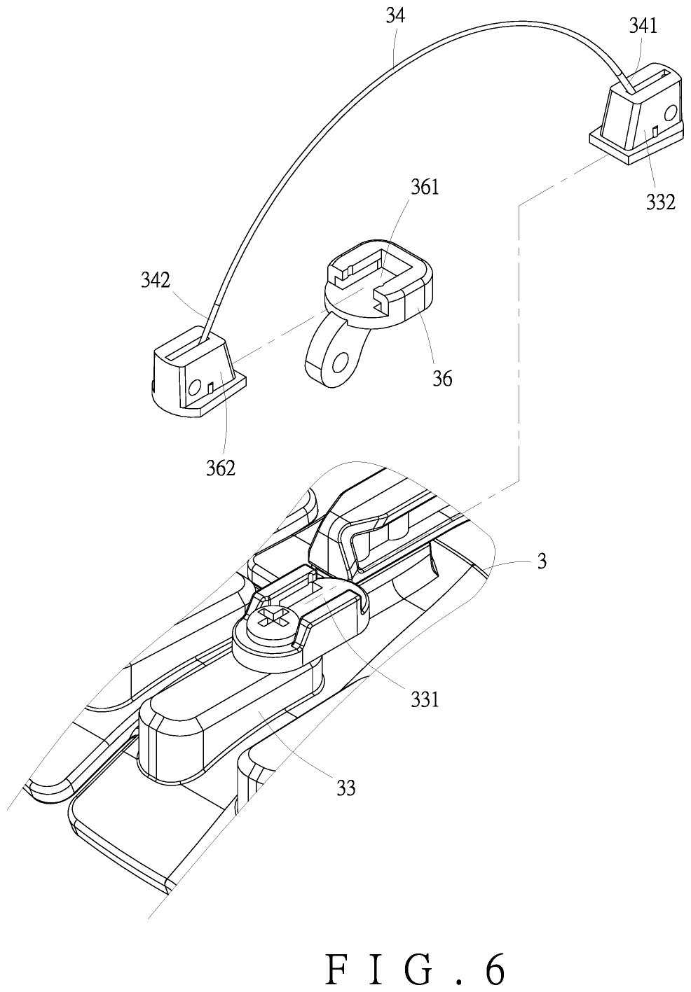

FIG. 6 illustrates a second embodiment. The second embodiment is substantially similar to the first embodiment with the exceptions described hereinafter.

A plurality of adjustment seats 33 are connected to the palm orthosis body 3. Each adjustment seat 33 is formed with a first locking groove 331. A first locking block 332 is detachably engaged in the first locking groove 331. A first end 341 of an elastic metal wire 34 is inserted and secured to the first locking block 332. A movable seat 36 is formed with a second locking groove 361. A second locking block 362 is detachably engaged in the second locking groove 361. A second end 342 of the elastic metal wire 34 is inserted and secured to the second locking block 362. When the first locking block 332 is separated from the first locking groove 331 and the second locking block 362 is separated from the second locking groove 361, the elastic metal wire 34 of a different diameter can be optionally replaced.

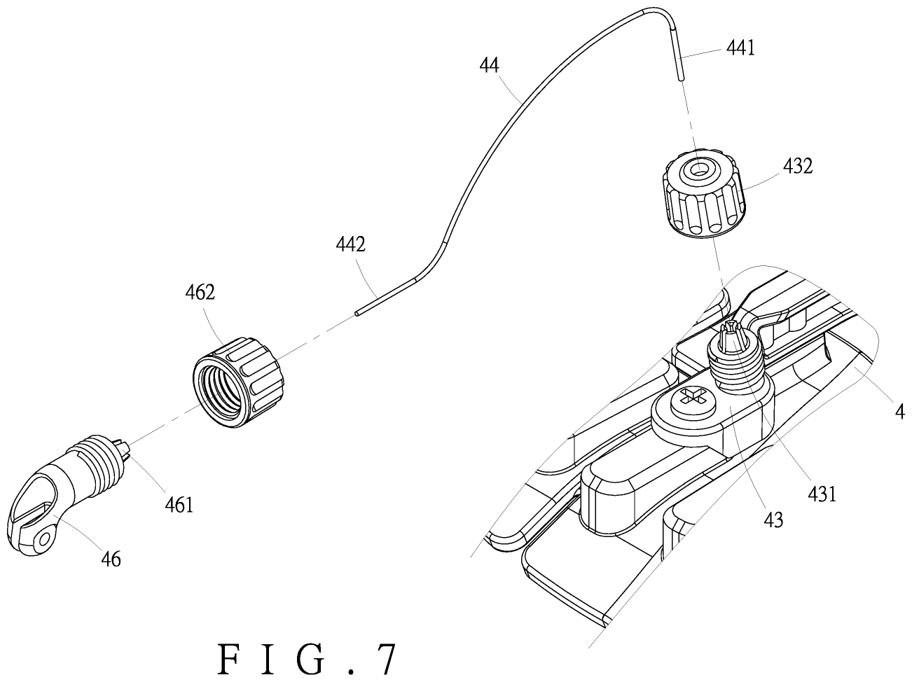

FIG. 7 illustrates a third embodiment. The third embodiment is substantially similar to the first embodiment with the exceptions described hereinafter.

A plurality of adjustment seats 43 are connected to the palm orthosis body 4. The adjustment seat 43 is provided with a first elastic portion 431 that is tapered and has a cut-out area. A first nut 432 is screwed to the first elastic portion 431. A first end 441 of an elastic metal wire 44 is inserted into the first elastic portion 431, and then the first nut 432 is tightened, so as to tightly secure the first end 441 of the elastic metal wire 44. A movable seat 46 is provided with a second elastic portion 461. A second nut 462 is screwed to the second elastic portion 461. A second end 442 of the elastic metal wire 44 is inserted into the second elastic portion 461, and then the second nut 462 is tightened, so as to tightly secure the second end 442 of the elastic metal wire 44. By unscrewing the first nut 432 and the second nut 462, the elastic metal wire 44 with a different diameter can be optionally replaced.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. An orthosis device having a metal wire adjusting structure, comprising:
a palm orthosis body, including a plurality of fixing portions, each of the fixing portions being detachably connected with an elastic metal wire, the elastic metal wire being further detachably connected to a movable seat, a finger sleeve being connected to the movable seat;
a forearm orthosis body, coupled to the palm orthosis body.

2. The orthosis device as claimed in claim 1, wherein each of the fixing portions is connected with an adjustment seat, the adjustment seat has a first through hole for insertion of a first end of the elastic metal wire, a first adjusting bolt is screwed to an outer side of the adjustment seat for tightening and securing the first end of the elastic metal wire, a second end of the elastic metal wire is inserted into a second through hole of the movable seat, and a second adjusting bolt is screwed to an outer side of the movable seat for tightening and securing the second end of the elastic metal wire.

3. The orthosis device as claimed in claim 1, wherein each of the fixing portions is connected with an adjustment seat, the adjustment seat is formed with a first locking groove, a first locking block is detachably engaged in the first locking groove, a first end of the elastic metal wire is inserted and secured to the first locking block, the movable seat is formed with a second locking groove, a second locking block is detachably engaged in the second locking groove, and a second end of the elastic metal wire is inserted and secured to the second locking block.

4. The orthosis device as claimed in claim 1, wherein each of the fixing portions is connected with an adjustment seat, the adjustment seat is provided with a first elastic portion that is tapered and has a cut-out area, a first nut is screwed to the first elastic portion, a first end of the elastic metal wire is inserted into the first elastic portion, the first nut is tightened to tightly secure the first end of the elastic metal wire, the movable seat is provided with a second elastic portion, a second nut is screwed to the second elastic portion, a second end of the elastic metal wire is inserted into the second elastic portion, and the second nut is tightened to tightly secure the second end of the elastic metal wire.

5. The orthosis device as claimed in claim 1, wherein the elastic metal wire is made of iron or steel.

6. The orthosis device as claimed in claim 1, wherein the elastic metal wire has a diameter of between 0.8 mm and 2.0 mm.

7. The orthosis device as claimed in claim 1, wherein the palm orthosis body is provided with a first coupling portion, the forearm orthosis body is provided with a second coupling portion, and the second coupling portion is coupled to the first coupling portion through a connecting rod.

8. The orthosis device as claimed in claim 1, wherein two sides of the forearm orthosis body are provided with fastening straps.

9. The orthosis device as claimed in claim 8, wherein the fastening straps are secured with a hook-and-loop tape.
